(19) United States
(12) Patent Application Publication
(10) Pub. No.: US 2004/0132190 A1
(43) Pub. Date: Jul. 8, 2004

(54) Gene therapy for myocardial ischemia

(75) Inventors: Wolfgang H. Dillmann, Solana Beach, CA (US); Frank J. Giordano, Del Mar, CA (US); Ruben Mestril, San Diego, CA (US)

Correspondence Address: QUINE INTELLECTUAL PROPERTY LAW GROUP, P.C., P O BOX 458, ALAMEDA, CA, 94501, US

(73) Assignee: The Regents of the University of California, Oakland, CA

(21) Appl. No.: 10/741,907

(22) Filed: Dec. 19, 2003

Related U.S. Application Data
(62) Division of application No. 09/664,127, filed on Sep. 18, 2000.
Application No. 09/664,127 is a continuation of application No. 09/098,174, filed on Jun. 16, 1998, now abandoned.
Application No. 09/098,174 is a continuation of application No. 08/660,387, filed on Jun. 7, 1996, now abandoned.
Application No. 08/660,387 is a continuation-in-part of application No. 08/481,122, filed on Jun. 7, 1995, now abandoned.
Application No. 08/481,122 is a continuation-in-part of application No. 08/396,207, filed on Feb. 28, 1995, now abandoned.

(51) Int. Cl. C12N 15/861; C12N 7/00

(52) U.S. Cl. 435/456; 435/320.1; 435/235.1

(57) ABSTRACT

The transgene-inserted replication-deficit adenoviral vector is effectively used in in vivo gene therapy for myocardial ischemia in a protective way, by a single intracoronary injection directly conducted deeply in the lumen of the coronary arteries in an amount sufficient for transfecting all cell types in the affected region, including cardiac myocytes.

This invention was made with Government support under Grant Nos. R01 HL-49343 and K14 HL-03150, awarded by the National Institute of Health. The Government has certain rights in this invention.

### FIELD OF THE INVENTION

The present invention relates to a recombinant adenovirus vector which is used in gene therapy for myocardial ischemia, a method for producing same, and a method of providing myocardial protection during revascularization or non-revascularization procedures with the use of the vector. The vector efficiently expresses a transgene in the myocardium.

### BACKGROUND OF THE ART

Myocardial ischemia occurs when the heart muscle does not receive an adequate blood supply and is thus deprived of necessary levels of oxygen and nutrients. The most common cause of myocardial ischemia is atherosclerosis, causing blockages in the blood vessels (coronary arteries) that provide blood flow to the heart muscle. Present treatment modalities include pharmacologic therapies coronary artery bypass surgery and percutaneous revascularization using techniques such as balloon angioplasty. In the setting of acute coronary occlusion (usually secondary to in-situ thrombosis of a coronary artery segment previously narrowed by atherosclerosis) treatment of acute myocardial ischemia is often achieved by using thrombolytic agents (“clot busters”) to open the occluded arteries. Standard pharmacologic therapy is predicated on strategies that involve either increasing blood supply to the heart muscle or decreasing the demand of the heart muscle for oxygen and nutrients. Increased blood supply to the myocardium is achieved by agents such as calcium channel blockers or nitroglycerin compounds that increase the diameter of diseased arteries by causing relaxation of the smooth muscle in the arterial walls. Decreased demand of the heart muscle for oxygen and nutrients is accomplished either by agents that decrease the hemodynamic load on the heart or those that decrease the contractile response of the heart to a given hemodynamic load. Surgical treatment of ischemic heart disease is based on the bypass of diseased arterial segments with strategically placed bypass graft (usually saphenous vein or internal mammary artery grafts). Percutaneous revascularization is based on the use of catheters to reduce the narrowing in diseased coronary arteries. All of these strategies are based on the eradication of ischemic episodes as the primary treatment evidence, and all have limitations of their effectiveness in this regard.

Australian Patent Publication No. 27902/92, corresponding to W093/06223, discloses adenovirus vectors for expression of desired genes in muscle cells to treat muscular dystrophy and thromboses. Although the '902 application discloses adenovirus type 5, it discloses specific vector constructs which are used for the treatment of muscular dystrophy. Texas Heart Institute Journal article 21:104-11 (1994) discloses the advantages of use of adenoviral vectors in mediating efficient direct gene transfer for preventing restenosis. In particular, this article teaches that the Ad5 virus transfected into 293 cells is an extremely useful vector for gene transfer in coronary arteries. Herz, 18(4):222-229 (1993) discloses the advantages of use of replication-deficient adenoviral vectors in direct gene therapy. Like the preceding article, this article teaches general advantages in use of the Ad5 virus. American Journal of Medical Science, 306(2):129-36 (1993) discloses the advantages of use of recombinant adenoviral vectors in gene transfer. This article teaches general advantages in use of adenoviral vectors, direct intravascular injection, and bFGF gene for treating coronary occlusion. However, all of the teachings of the above documents are too general to address the in vivo expression efficiency of a certain vector in myocardial protection.

In particular, none of the treatment modalities of the prior art addresses the issue of protection of the myocardium against irreversible damage when ischemia does occur. Protection of heart muscle against ischemia has been demonstrated in the setting of ischemic pre-conditioning. This phenomenon occurs when the heart is exposed to brief periods of ischemic stress prior to a prolonged ischemic episode. During the brief periods of ischemia, production of specific stress related factors is induced. These stress factors protect the myocardium against subsequent and potentially more harmful, prolonged ischemic episodes. To date, attempts to induce these same factors by pharmacologic means have been unsuccessful.

### BRIEF DESCRIPTION OF THE FIGURES

FIG. 1 is a schematic figure which shows rescue recombination construction of a transgene encoding adenovirus.

FIG. 2 schematically presents the strategy for introducing a foreign gene into the E1 region of a replication-deficient adenoviral vector.

FIG. 3 graphically presents the lactate dehydrogenase released by adenoviral infected H9c2 cells following simulated ischemia.

FIG. 4 graphically presents the creatine kinase released by adenoviral infected neonatal rat myocytes following simulated ischemia.

### SUMMARY OF THE INVENTION

The present invention has exploited a gene therapy approach to treat heart disease. An objective of the present invention is to provide a method of providing myocardial protection in which a stress related factor is produced in the myocardium and is present at the time of ischemia so as to protect the myocardium against subsequent, potentially more harmful, prolonged ischemic episodes. This objective concerns protective effects, rather than therapeutic effects on myocardial ischemia.

Namely, one important aspect of the present invention is a method of providing myocardial protection, comprising: delivering a replication-deficient adenoviral vector to a myocardium by intracoronary injection into the coronary arteries, preferably a single injection of the vector, directly into the coronary arteries, so as to transfect cardiac myocytes, which do not undergo rapid turnover, in the affected myocardium, said vector comprising a transgene coding for a stress related factor such as heat shock proteins HSP70i, HSP27, HSP40 and HSP60, and the adenosine A3 receptor; and expressing the transgene in the myocardium, thereby raising the level of stress related factor in the affected region of the myocardium. By injecting the vector stock containing no wild-type virus deeply into the lumen of the coronary arteries, preferably into both the right and left coronary arteries, of the myocardium preferably in an ischemic milieu, preferably in an amount of 10¹⁰-10¹³ viral particles as determined by optical densitometry (more preferably 10¹¹-10¹² viral particles), it is possible to locally transfect most of the cells, especially cardiac myocytes, which do not undergo rapid turnover, in the affected myocardium with the genes for a stress related factor, thereby maximizing myocardial protection efficacy of gene transfer, and minimizing the possibility of an inflammatory response to viral proteins. If a ventricular myocyte-specific promoter is used, the promoter more securely enables expression limited to the cardiac myocytes so as to effectively avoid the potentially harmful effects of angiogenesis in non-cardiac tissues such as the retina.

In the above method, myocardial protection is expected to be more effective in cases that (a) said patient has non-revascularized ischemic heart disease and said protection is desired during planned non-cardiac surgery, wherein said vector is administered a plurality of days prior to the planned non-cardiac surgery; (b) said protection is desired in anticipation of complex percutaneous revascularization, and wherein said vector is delivered at the time of a diagnostic catheterization a plurality of days prior to the revascularization; (c) said protection is desired in anticipation of complex cardiac surgery, and wherein said vector is delivered at the time of a diagnostic cardiac catheterization; (d) said protection is desired in a donor heart to be transplanted into a host patient with a coronary disease, and herein said vector is delivered at the time of a diagnostic coronary angiography prior to explanation to rule out coronary disease; and (e) said protection is desired in a patient with diffuse, nonrevascularizable coronary artery disease, at the time of a diagnostic coronary angiography prior to explanation to rule out coronary disease, wherein said vector is delivered a plurality of times.

Another aspect of the present invention is an injectable adenoviral vector preparation, comprising a recombinant adenoviral vector, preferably in a final viral titer of 10¹⁰-10¹² viral particles, said vector containing no wild-type virus and comprising a partial adenoviral sequence from which the E1A/E1B genes have been deleted, and a transgene coding for a stress related factor such as heat shock proteins HSP70i HSP27, HSP40 and HSP60, and the adenosine A3 receptor, driven by a promoter flanked by the partial adenoviral sequence; and a pharmaceutically acceptable carrier. By using this injectable adenoviral vector preparation, it is possible to perform effective adenovirus-mediated stress related factor-coding gene transfer for the treatment of clinical myocardial ischemia without any undesirable effects.

A further aspect of the present invention is a method of production of a viral stock containing a recombinant vector capable of expressing a stress related factor in vivo in the myocardium, comprising the steps of cloning a transgene coding for a stress related factor such as heat shock proteins HSP70i HSP27, HSP40 and HSP60, and the adenosine A3 receptor into a plasmid containing a promoter and a polylinker flanked by partial adenoviral sequences of the left end of the human adenovirus 5 genome from which the E1A/E1B genes have been deleted; co-transfecting said plasmid into mammalian cells transformed with the E1A/E1B genes, with a plasmid which contains the entire human adenoviral 5 genome and an additional insert making the plasmid too large to be encapsulated, whereby rescue recombination takes place between the transgene-inserted plasmid and the plasmid having the entire adenoviral genome so as to create a recombinant genome containing the transgene without the E1A/E1B genes, said recombinant genome being sufficiently small to be encapsidated; identifying successful recombinants in cell cultures; propagating the resulting recombinant in mammalian cells transformed with the E1A/E1B genes; and purifying the propagated recombinants so as to contain the recombinant vector, without wild-type virus therein.

Based on the present invention, effective protection to the heart muscle against myocardial ischemia such as that encountered during threatened myocardial infarction (heart attack) can be surprisingly achieved. That is, the present invention allows for protection against tissue necrosis secondary to prolonged ischemic episodes. Technical details are delineated below.

### DETAILED DESCRIPTION OF THE PREFERRED EMBODIMENT

Transgenes Coding for Stress Related Factors

In the present invention, various stress related factors which are capable of protecting myocardial ischemia can be used; heat shock proteins HSP70i, HSP27, HSP40 and HSP60, and the adenosine A3 receptor can be exemplified. Adenosine plays an important role in mediating the phenomenon of ischemic preconditioning. The function of adenosine appears to be mediated via A3 type adenosine receptors. In cell culture experiments in which the number of A3 receptors per cell was increased, the efficacy of an adenosine analogue (Gensia Pharmaceutical) to mitigate protection against ischemia was increased. The coding regions for these factors are known in the art, and it is possible to download these cDNA sequences from Genebank and other databanks over the internet, for example. Full or partial length cDNAs coding for the above factors can be used in the present invention. Other than above, sarcoplasmic reticular calcium ATPase can be used for the purpose of studying myocardial calcium handling/hypertrophy.

Helper Independent Replication Deficient Human Adenovirus 5 System

The cDNA of interest is transferred to the myocardium, including cardiac myocytes, in vivo and directs constitutive production of the re-encoded protein. Viral vectors provide a means for highly efficient gene transfer. Several different gene transfer approaches are feasible. The present inventors initially used the helper-independent replication deficient human adenovirus 5 system which has previously demonstrated transfection greater than 60% of myocardial cells in vivo by a single intracoronary injection. Non-replicative recombinant adenoviral vectors are particularly useful in-transfecting coronary endothelium and cardiac myocytes resulting in highly efficient transfection after intravenous injection. The recombinant adenoviral vectors based on the human adenovirus 5 {Virology, 163:614-617 (1988)} are missing essential early genes from the adenoviral genome (usually E1A/E1B), and are therefore unable to replicate unless grown in “permissive” cell lines that provide the missing gene products in trans. In place of the missing adenoviral genomic sequences, a transgene of interest can be cloned and will be expressed in tissue/cells infected with the replication deficient adenovirus. Although adenovirus-based gene transfer does not result in integration of the transgene into the host genome (less than 0.1% adenovirus-mediated transfections result in transgene incorporation into host DNA), and therefore is not stable, adenoviral vectors can be propagated to high titer and allow gene transfer to non-replicating cells. Although the transgene is not passed to daughter cells, in the case of the adult cardiac myocytes, which do not divide, this is not an important limitation. Retroviral vectors provide stable gene transfer, and high titers are now obtainable via retrovirus pseudotyping {Burns, et al., Proc. Natl. Acad. Sci. (USA), 90:8033-8037 (1993)}, but current retroviral vectors are unable to traduce nonreplicating cells (adult cardiac myocytes) efficiently. In addition, the potential hazards of transgene incorporation into host DNA are not warranted if short-term gene transfer is sufficient. Thus, a limited duration expression of a stress related factor may be sufficient for temporary myocardial protection, and transient gene transfer for some cardiovascular disease processes may be adequate and possibly preferable.

Human 293 cells, which are human embryonic kidney cells transformed with adenovirus E1A/E1B genes, typify the permissive cell lines. However, other cell fines which allow replication-deficient adenoviral vectors to propagate therein can be used. Thus, other cell lines useful for this purpose include HeLa cells.

Construction of Recombinant Adenoviral Vectors

All adenoviral vectors used in the present invention can be constructed by the rescue recombination technique developed by Frank Graham {Virology, 163:614-617 (1988)}. Briefly, the transgene of interest is cloned into a shuttle vector that contains a promoter, polylinker and partial flaking adenoviral sequences from which E1A/E1B genes have been deleted. As the shuttle vector, plasmid pACI (Virology, 163:614-617 (1988)} (or an analog) which encodes portions of the left end of the human adenovirus 5 genome {Virology, 163:614-617 (1988)} minus the early protein encoding E1A and E1B sequences that are essential for viral replication, and plasmid ACCMVPLPA {J. Biol. Chem., 267:25129-25134 (1992)} which contains polylinker, the CMV promoter and SV40 polyadenylation signal flanked by partial adenoviral sequences from which the E1A/E1B genes have been deleted can be exemplified. The use of plasmid pAC1 or ACCMVPLA facilitates the cloning process. The shuttle vector is then co-transfected with a plasmid which contains the entire human adenoviral 5 genome with a length too large to be encapsidated, into 293 cells. Co-transfection can be conducted by calcium phosphate precipitation or lipofection under conditions such as those disclosed by Biotechniques 15:868-872 (1993). As the plasmid having the entire adenoviral 5 genome, plasmid JM17 which encodes the entire human adenovirus 5 genome plus portions of the vector pBR322 including the gene for ampicillin resistance (4.3 kb) is exemplified. Although JM17 encodes all of the adenoviral proteins necessary to make mature viral particles, it is too large to be encapsidated (40 kb versus 36 kb for wild type). In a small subset of co-transfected cells, rescue recombination between the transgene containing the shuttle vector such as plasmid pAC1 and the plasmid having the entire adenoviral 5 genome such as plasmid pJM17 takes place so as to create a recombinant genome that is deficient in the E1A/E1B sequences, and that contains the transgene of interest but secondarily loses the additional sequence such as the pBR322 sequences during recombination, thereby being small enough to be encapsidated (see FIG. 1). With respect to the above method, we have reported successful results {Giordano, et al., Circulation, 88:1-139 (1993) and Giordano and Hammond, Clin. Res., 42:123A (1994)}. The CMV driven β-galactosidase encoding adenovirus HCMVSP1LacZ {Clin. Res. (Abs), 42:123A (1994)} can be used to evaluate efficiency of gene transfer using X-gal treatment.

The initial mode of gene transfer uses adenoviral vectors as delineated above. The advantages of these vectors include the ability to effect high efficiency gene transfer (more than 60% of target organ cells transfected in vivo), the ease of obtaining high titer viral stocks and the ability of these vectors to effect gene transfer into cells such as cardiac myocytes which do not undergo rapid turnover. One potential disadvantage is that the current generation of this vector does not result in stable gene transfer. Genes transferred to the myocardium by adenovirus vectors do not integrate into the host cell DNA and, therefore, do not get passed on to the progeny of dividing cells (fibroblasts, endothelial cells, smooth muscle cells, etc.). Genes transferred to the myocardium by current generation adenoviral vectors remain active only for a period of weeks to months. This may actually be advantageous for certain clinical applications such as myocardial protection to induce a controlled amount of a stress related factor.

Alternatively, newer generation adenoviral vectors that have further deletions in the adenovirus genome (in addition to E1A/E1B) are under development. These vectors have the potential to effect longer term gene transfer and to be less immunogenic. If it is determined that longer term gene transfer would be more efficacious and/or inflammatory response to first generation vectors becomes problematic, these newer generation vectors could be used. In addition, if gene transfer limited to the arterial wall proves as efficacious as myocardial gene transfer to effect myocardial protection, alternative method of gene transfer could be used including electroporation, use of hydrogel coated balloon catheters, use of liposomes or use of alternate viral vectors including retrovirus or adeno associated viral vectors.

Cardiac-Specific Promoters

It is also proposed in the present invention to use cell targeting not only by delivery of the transgene into the coronary artery, but also, in additional experiments, by using a ventricular myocyte-specific promoter. By fusing the tissue-specific transciptional control sequences of left ventricular myosin light chain-2 (MLC₂ᵥ) to a transgene such as the FGF-5 gene within the adenoviral construct, transgene expression is limited to ventricular cardiac myocytes. The efficacy of gene expression and degree of specificity provided by the MLC₂ᵥ promoter with lacZ have been determined, using the recombinant adenoviral system of the present invention. Cardiac-specific expression has been documented previously by Lee, et al. {J. Biol. Chem., 267:15875-15885 (1992)}. The MLC₂ᵥ promoter is comprised of 250 bp, and easily fits within the adenoviral-5 packaging constraints. The myosin heavy chain promoter, known to be a vigorous promoter of transcription, cannot be used because its large size (5.5 kb) cannot fit within the adenoviral vector. Other promoters, such as the troponin-C promoter, while highly efficacious and sufficiently small, lacks adequate tissue specificity. By using the MLC₂ᵥ promoter and delivering the transgene in vivo, it is believed that the cardiac myocyte alone (that is without concomitant expression in endothelial cells, smooth muscle cells, and fibroblasts within the heart) will provide adequate expression of a stress related factor such as heat shock proteins HSP70i HSP27, HSP40 and HSP60, and the adenosine A3 receptor to promote myocardial protection. Limiting expression to the cardiac myocyte also has advantages regarding the utility of gene transfer for the treatment of clinical myocardial ischemia. By limiting expression to the heart, one avoids any potentially harmful effect in non cardiac tissues. In addition, of the cells in the heart, the myocyte would likely provide the longest transgene expression since the cells do not undergo rapid turnover; expression would not therefore be decreased by cell division and death as would occur with endothelial cells. Subsequent studies will determine whether targeting gene expression to the endothelial cells, and limiting expression somewhat to the coronary endothelium by intracoronary injection, will be a sufficient means to deliver the transgene. Endothelial-specific promoters are already available for this purpose {Lee, et al., J. Biol. Chem., 265:10446-10450 (1990)}. As yet there are no fibroblast or smooth muscle cell promoters available that would efficiently limit expression of the transgene to smooth muscle or fibroblasts within the heart.

In the present invention, targeting the heart by intracoronary injection with a high titer of the vector, and transfecting all cell types can maximize the probability for success. Namely, it is believed that a more dramatic result can be achieved if not only myocytes but also cell types other than myocytes are targeted as well, although they are dividing cells.

Propagation and Purification of Adenoviral Vectors

Successful recombinant vectors can be plaque purified according to standard methods. The resulting viral vectors are propagated on 293 cells which provide E1A and E1B functions in trans to titers in the 10¹⁰-10¹² viral particles/ml range. Cells can be infected at 80% confluence and harvested at 36-48 hours post-infection. After 3 freeze-thaw cycles the cellular debris is pelleted by standard centrifugation and the virus further purified by CsCl gradient ultracentrifugation (double CsCl gradient ultracentrifugation is preferred). Prior to in vivo injection, the viral stocks are desalted by gel filtration through sepharose columns such as G25 sephadex. The resulting viral stock has a final viral titer in the range of 10¹⁰-10¹² viral particles/ml. The adenoviral construct must be highly purified, with no wild-type (potentially replicative) virus. Impure constructs can cause an intense immune response in the host animal. From this point of view, propagation and purification must be conducted to exclude contaminants and wild-type virus by, for example, identifying successful recombinants with PCR using appropriate primers, conducting two rounds of plaque purification, and double CsCl gradient ultracentrifugation.

Delivery of Recombinant Adenoviral Vectors

The viral stock can be in the form of an injectable preparation containing pharmaceutically acceptable carrier such as saline, as necessary. The final titer of the vector in the injectable preparation is preferably in the range of 10¹⁰-10¹² viral particles which allows for effective gene transfer. The adenovirus transgene constructs are delivered to the myocardium by direct intracoronary injection using standard percutaneous catheter based methods under fluoroscopic guidance, at an amount sufficient for the transgene to be expressed to a degree which allows for highly effective therapy. The amount of the vector to be injected is preferably in the range of 10¹⁰-10¹³ viral particles (more preferably 10¹¹-10¹² viral particles). The injection should be made deeply (such as 1 cm within the arterial lumen) into the lumen of the coronary arteries, and preferably be made in both coronary arteries, as the growth of collateral blood vessels is highly variable within individual patients. By injecting the material directly into the lumen of the coronary artery by coronary catheters, it is possible to target the gene rather effectively, and to minimize loss of the recombinant vectors to the proximal aorta during injection. Gene expression when delivered in this manner is minimal in the liver, and viral RNA cannot be found in the urine at any time after intracoronary injection. Any variety of coronary catheter, or a Stack perfusion catheter, and so forth can be used in the present invention.

Protective Applications

The replication deficient recombinant adenoviral vectors of the present invention allow for highly efficient gene transfer in vivo without evidence for cytopathic effect or inflammation in the areas of gene delivery. Based on these results, it is believed that a high enough degree of in vivo gene transfer to effect in vivo functional changes is achieved. In particular, protective use of the vectors can be advantageous. In order to provide optimal protection to the myocardium, stress proteins must be present at the time of ischemia. This requires gene transfer prior to anticipated ischemia. Although the timing of many prolonged ischemic episodes is unpredictable, there are specific settings during which ischemia is anticipated. These circumstances specifically allow for gene transfer prior to the ischemic event. The following include some of the clinical settings in which a role for a therapeutic gene transfer approach is anticipated:

1. Gene transfer to provide myocardial protection during noncardiac surgery in patients with non-revascularized ischemic heart disease. This is common clinical problem which often requires a coronary revascularization procedure (e.g., angioplasty or bypass surgery) before proceeding with the non-cardiac surgery (hip replacement, gall bladder surgery, etc.). If revascularization is not possible because the coronary vasculature is diffusely diseased or the risk of cardiac surgery is thought to be unacceptably high, the non-cardiac surgery is often precluded thus exposing the patient to further morbidity. In these settings, gene transfer could be effected by intracoronary injection of the viral construct several days prior to the planned non-cardiac surgery such that levels of protective stress factors in the myocardium would be high during the anticipated surgery. Cardiac catheterization, necessary for gene delivery, does not require anesthesia and is very well tolerated by otherwise clinically compromised patients.

2. Gene transfer to provide myocardial protection during complex percutaneous revascularization procedures (angioplasty, atherectomy, etc.) during which prolonged ischemia is anticipated. Percutaneous revascularization of the coronary vasculature is complicated 4% of the time by abrupt total closure of the target vessel. Although ischemia can often be aborted by use of intracoronary thrombolytic agents, placement of intracoronary stents or emergent bypass surgeries, frequently associated with irreversible myocardial damage. Even when abrupt vessel closure does not occur, a significant number of procedures are complicated by “slow-flow” secondary to non-occlusive in situ thrombosis or micro-embolization to the distal coronary vasculature (common when treating diseased bypass grafts). These patients are also at high risk of peri-procedural myocardial damage. Usually, these patients undergo a diagnostic cardiac catheterization several days prior to percutaneous revascularization. Intracoronary gene delivery to the myocardium at risk at the time of the diagnostic catheterization in anticipation of revascularization in high risk patients is believed to be conspicuously effective.

3. Gene transfer to provide myocardial protection during complex cardiac surgery (complex revascularization procedures, valve surgery, complex congenital heart corrective surgeries, etc.) Coronary artery bypass surgery is associated with a 3-6.5% incidence of peri-operative myocardial infarction. When peri-operative infarction does occur, peri-operative mortality is higher and in patients with residual left ventricular function and incomplete revascularization the long-term prognosis is poorer. Gene transfer by intracoronary injection at the time of diagnostic cardiac catheterization just prior to surgery is believed to be especially effective. We anticipated this approach would be helpful both in high-risk valve surgery and congenital heart disease surgeries.

4. Gene transfer to provide myocardial protection to donor hearts prior to cardiac transplantation. Damage to donor hearts as a result of unavoidable delays between the time of explanation and the time of grafting into the host patient is responsible for a significant proportion of transplant related morbidity and failed transplantation procedures. Donor hearts often undergo diagnostic coronary angiography prior to explanation in order to rule out coronary disease. Gene transfer to the myocardium by intracoronary injection at this time is believed to be particularly effective.

5. Gene transfer to provide myocardial protection to patients with diffuse, nonrevascularizable coronary artery disease. A subset of patients with coronary artery disease cannot be safely revascularized. This subset includes patients with diffuse coronary disease in whom bypass surgery is technically not feasible, and patients with preclusive co-morbidity such as severe lung disease. In these patients, long-term gene transfer to protect the myocardium against chronic recurrent ischemia is believed to be particularly effective.

Animal Model of Myocardial Ischemia

Important prerequisites for successful studies on gene therapy are (a) constitution of an adequate animal model which is applicable to myocardial ischemia of an enormous patient population, and which can provide useful data regarding mechanisms for myocardial protection in the setting of myocardial ischemia, and (b) accurate evaluation of the effects of gene transfer. From this point of view, none of the prior art is satisfactory. It is proposed in the present invention to use a porcine model of myocardial ischemia that mimics clinical coronary artery disease. Placement of an ameroid constrictor around the left circumflex (LCx) coronary artery results in gradual complete closure (within 7 days of placement) with minimal infarction (1% of the left ventricle, 4±1% of the LCx bed) {Roth, et al., Circulation, 82:1778 (1990), Roth, et al., Am. J. Physiol., 235:H1279 (1987), White, et al., Circ. Res., 71:1490 (1992), Hammond, et al., Cardiol., 23:475 (1994), and Hammond, et al., J. Clin. Invest., 92:2644 (1993)}. Myocardial function and blood flow are normal at rest in the region previously perfused by the occluded artery (referred to as the ischemic region), due to collateral vessel development, but blood flow reserve is insufficient to prevent ischemia when myocardial oxygen demands increase. Thus, the LCx bed is subject to episodic ischemia, analogous to clinical angina pectoris. Collateral vessel development and flow-function relationships are stable within 21 days of ameroid placement, and remain unchanged for four months {Roth, et al., Circulation, 82:1778 (1990), Roth, et al., Am. J. Physiol., 235:H1279 (1987), White, et al., Circ. Res. 71:1490 (1992)}. It has been documented by telemetry that animals have period ischemic dysfunction in the bed at risk throughout the day, related to abrupt increases in heart rate during feeding, interruptions by personnel etc. (unpublished data). Thus, the model has a bed with stable but inadequate collateral vessels, and is subject to periodic ischemia. Another distinct advantage of the model is that there is a normally perfused and functioning region (the LAD bed) adjacent to an abnormally perfused and functioning region (the LCx bed), thereby offering a “control” bed within each animal.

Myocardial contrast echocardiography can be used to estimate regional myocardial perfusion in the present invention. The contrast material is composed of microaggregates of galactose and increases the echogenicity (“whiteness”) of the image. The microaggregates distribute into the coronary arteries and myocardial walls in a manner that is proportional to blood flow {Skyba, et al., Circulation, 90:1513-1521 (1994)}. Although it is difficult to obtain precise quantitative information with this technique, it has been shown that peak intensity of contrast is closely correlated with myocardial blood flow as measured by microspheres {Skyba, et al., Circulation, 90:1513-1521 (1994)}. Since the echocardiographic images can accurately identify the LCx bed, and myocardial contrast echocardiography can be used to evaluate myocardial blood flow, a hydraulic cuff occluder can be placed around the proximal LCx adjacent to the ameroid.

PCR can be used to detect stress related factor DNA and mRNA in myocardium from animals that has received gene transfer. In addition, two weeks after gene transfer, myocardial samples from all five lacZ-infected animals show substantial β-galactosidase activity on histological inspection. In addition, using a polyclonal antibody to a stress related factor such as heat shock protein expressed in cells and in myocardium from animals that have received gene transfer can be demonstrated.

### EXPERIMENT 1

### Adenoviral Constructs

A helper independent replication deficient human adenovirus 5 system was used. The genes of interest were lacZ and FGF-5. The full length cDNA for human FGF-5 was released from plasmid pLTR122E {Zhen, et al., Mol. Cell. Biol., 8:3487 (1988)} as a 1.1 kb ECOR1 fragment which includes 981 bp of the open reading frame of the gene, and cloned into the polylinker of plasmid ACCMVPLPA which contains the CMV promoter and SV40 polyadenylation signal flanked by partial adenoviral sequences from which the E1A and E1B genes (essential for viral replication) had been deleted. This plasmid was co-transfected (lipofection) into 293 cells with plasmid JM17 which contained the entire human adenoviral 5 genome with an additional 4.3 kb insert making pJM17 too large to be encapsidated. Homologous rescue recombination resulted in adenoviral vectors containing the transgene in the absence of E1A/E1B sequences. Although these recombinants were nonreplicative in mammalian cells, they could propagate in 293 cells which had been transformed with E1A/E1B and provided these essential gene products in trans. Transfected cells were monitored for evidence of cytopathic effect which usually occurred 10-14 days after transfection. To identify successful recombinants, cell supernatant from plates showing a cytopathic effect was treated with proteinase K (50 mg/ml with 0.5% sodium dodecyl sulfate and 20 mM EDTA) at 56° C. for 60 minutes, phenol/chloroform extracted and ethanol precipitated. Successful recombinants were then identified with PCR using primers {Biotechniques, 15:868-872 (1993)} complementary to the CMV promoter and SV40 polyadenylation sequences to amplify the insert (the expected 1.1 kb fragment), and primers {Biotechniques, 15:868-872 (1993)} designed to concomitantly amplify adenoviral sequences. Successful recombinants then underwent two rounds of plaque purification. Viral stocks were propagated in 293 cells to titers ranging between 10¹⁰ and 10¹² viral particles, and were purified by double CsCl gradient centrifugation prior to use. Recombinant adenoviruses encoding β-galactosidase, or HSP70i were constructed using full length cDNAs. The system used to generate recombinant adenoviruses imposed a packing limit of 5 kb for transgene inserts. The genes proposed, driven by the CMV promoter and with the SV40 polyadenylation sequences were less than 4 kb, well within the packaging constraints. Recombinant vectors were plaque purified by standard procedures. The resulting viral vectors were propagated on 293 cells to titers in the 10¹⁰-10¹² viral particles range. Cells were infected at 80% confluence and harvested at 36-48 hours. After freeze-thaw cycles the cellular debris was pelleted by standard centrifugation and the virus further purified by double CsCl gradient ultracentrifugation (discontinuous 1.33/1.45 CsCl gradient; cesium prepared in 5 mM Tris, 1 mM EDTA (pH 7.8); 90,000×g (2 hr), 105,000×g (18 hr)). Prior to in vivo injection, the viral stocks were desalted by gel filtration through sepharose columns such as G25 sephadex. The resulting viral stock had a final viral titer in the 10¹⁰-10¹² viral particles range. The adenoviral construct was highly purified, with no wild-type (potentially replicative) virus.

### EXPERIMENT 2

### Adult Rat Cardiomyocytes in Cell Culture

Adult rat cardiomyocytes were prepared by Langendorf perfusion with a collagenase containing perfusate according to standard methods. Rod shaped cells were cultured on laminin coated plates and at 24 hours were infected with the β-galactosidase-encoding adenovirus obtained in the above Experiment 1 at a multiplicity of infection of 1:1. After a further 36 hour period the cells were fixed with glutaraldehyde and incubated with X-gal.

Consistently 70-90% of adult myocytes expressed the β-galactosidase transgene after infection with the recombinant adenovirus. At a multiplicity of infection of 1-2:1 there was no cytotoxicity observed.

### EXPERIMENT 3

### Pig Myocardium In Vivo

The β-galactosidase-encoding adenoviral vector obtained in the above Experiment 1 was propagated in permissive 293 cells and purified by CsCl gradient ultracentrifugation with a final viral titer of 1.5×10¹⁰ viral particles, based on the procedures of Experiment 1. An anesthetized, ventilated 40 kg pig underwent thoracotomy and isolation of the left circumflex and left anterior descending coronary arteries. A 26 gauge butterfly needle was inserted in the mid left anterior descending (LAD) coronary artery and the vector (1.5×10¹⁰ viral particles) was injected in a 2 ml volume. The chest was closed and the animal allowed to recover. On the fourth post-injection day the animal was sacrificed. The heart fixed with perfused glutaraldehyde, sectioned and incubated with X-gal for 16.5 hours. After imbedding and sectioning the tissue was counterstained with eosin.

Microscopic analysis of tissue sections (transmural sections of LAD bed 72 hours after intracoronary injection of adenovirus containing lacZ) revealed a significant magnitude of gene transfer observed in the LAD coronary bed with many tissue sections demonstrating greater than 50-60% of the cells staining positively for β-galactosidase. Areas of the myocardium remote from the LAD circulatory bed did not demonstrate X-gal staining and served as a negative control, while diffuse expression of a gene was observed in myocytes and in endothelial cells. The majority of myocytes showed β-galactosidase activity (blue stain), and, in subsequent studies using closed-chest intracoronary injection, similar activity was present 14 days after gene transfer (n=6). There was no evidence of inflammation or necrosis in the areas of transfection.

### EXPERIMENT 4

### Pig Constriction Model

Animals and Instrumentation

Details are based on previous studies {Hammond, et al., J. Clin. Invest., 92:2644-2652 (1993) and Roth, et al., J. Clin. Invest., 91:939-949 (1993)}. Animals includes 14 domestic pigs, (30-40 kg). A left thoracotomy is performed under sterile conditions for instrumentation. Catheters are placed in the left atrium and aorta, providing a means to measure regional blood flow, and to monitor pressures. Wires are sutured on the left atrium to permit ECG recording and atrial pacing. Finally, an ameroid is placed around the proximal LCx. After a stable degree of ischemia has developed, this treatment group (n=8) receives an adenoviral construct that includes genes for HSP70i (a heat shock protein), driven by a CMV promoter. Control animals (n=5) receives gene transfer with an adenoviral construct that includes a reporter gene, lacZ, driven by a CMV promoter.

Adenoviral Constructs

The helper independent replication deficient human adenovirus 5 system constructed in the above Experiment 1 is used. The genes of interest are lacZ and hsp70i. The material injected in vivo is highly purified and contains no wild-type (replication competent) adenovirus. Thus the possible in vivo adenoviral infection and inflammatory infiltration in the heart are minimized. By injecting the material directly into the lumen of the coronary artery by coronary catheters, it is possible to “target” the gene rather effectively. Gene expression when delivered in this manner in minimal in the liver, and viral RNA cannot be found in the urine at any time after intracoronary injection.

Delivery of the Transgene

Techniques for large animal surgery are described in Hammond, et al., J. Clin. Invest., 92:2644-2652 (1993), Hammond, et al., J. Amer. Coll. Cardiol., 23:475-482 (1994), Roth, et al., J. Clin. Invest., 91:939-949 (1993), and Ping, et al., Am. J. Physiol., 267:H2679 (1994). Injection of the construct (4.0 ml containing 10¹¹ viral particles of adenovirus) is made by injecting 2.0 ml into both the left and right coronary arteries (collateral flow to the LCx bed appeared to come from both vessels). Animals are anesthetized, and arterial access acquires via the right carotid by cut-down; a 5F Cordis sheath is placed. A 5F Multipurpose (A2) coronary catheter is used to engage the coronary arteries. Closure of the LCx ameroid is confirmed by contrast injection into the left main coronary artery. The catheter tip is then placed 1 cm within the arterial lumen so that minimal material will be lost to the proximal aorta during injection. This procedure is carried out for each of the pigs.

Assessment of Myocardial Protection

The strategy for myocardial protective studies include the timing of transgene delivery, the route of administration of the transgene, and choice of the stress related gene, using the aforesaid construct including a reporter gene (lacZ) and that including a stress related factor gene as well as the aforesaid pig models. The ameroid model of myocardial ischemia is chosen, and gene transfer is performed after stable. Gene transfer are effected by intracoronary injection of the viral construct several days prior to non-cardiac surgery or a diagnostic cardiac catheterization such that levels of protective stress factors in the myocardium will be high during the anticipated surgery or percutaneous revascularization. In addition, gene transfer by intracoronary injection is conducted at the time of diagnostic cardiac catheterization just prior to surgery. Myocardial protection can be assessed by the aforesaid echocardiography and microscopic analysis.

### EXPERIMENT 5

### Adenovirus Mediated Gene Transfer of a Heat Shock

Protein 70 (HSP70i) Protects Against Simulated Ischemia

In the following experiment, applicants inserted the heat shock protein 70 gene into an adenoviral vector and showed that they could infect neonatal rat cardiomyocytes and the myogenic rat cell line H9c2, and could further achieve very high levels of expression of the introduced gene (hsp70i).

Moreover, the cells infected with the adenoviral-hsp70i construct were also rendered tolerant to simulated ischemia as compared to cells infected with a control recombinant adenoviral construct.

The experiment showed that the adenovirus mediated transfer of hsp70i is not only efficient, but also highly effective in providing protection against simulated ischemic injury. The following describes the experiment in detail.

### MATERIALS AND METHODS

### Cell Culture

Neonatal rat cardiomyocytes were cultured as previously described {Iwaki, et al., Circulation, 87:2023-2032 (1993)}. The embryonic rat heart-derived cell line H9c2(2-1) and the human embryonic kidney cell line 293 were both obtained from the American Type Culture Collection, Rockville, Md., and were maintained in DMEM supplemented with antibiotics (penicillin/streptomycin/fungizone) and 10% fetal calf serum (FCS). Cells were infected in 60 cm tissue culture plates at about 80% confluency by adding enough of the adenoviral infectious stock to 1 ml of DMEM containing 2% heat inactivated FCS. To obtain a multiplicity of infection (MOI) of 10:1 or 1:1, cells were incubated with viral constructs for 60 minutes with mild constant shaking, 2 ml of DMEM/2% heat-inactivated FCS was then added and the plates incubated for 2 days in a 37° C., CO₂ incubator. Simulated ischemia of the infected neonatal rat cardiomyocytes and H9c2 plates were done as previously described {Mestril, et al., J. Clin. Invest., 98:759-767 (1994), hereby incorporated by reference in its entirety)}.

### Construction of Replication-Deficient Adenoviral Vectors

The inducible rat hsp70 described previously {Mestril, et al., Biochem. J., 298:561-569 (1994)} was inserted into the E1 region of an adenoviral vector construct using the general strategy previously described in Graham and Prevec, “Manipulation of Adenovirus Vectors”, in Methods in Molecular Biology, Vol. 7, pp 109-128, Murray, E. J. (eds), The Humana Press, Clifton, N.J. (1991). Briefly, the rat hsp70 gene was cloned into the multiple cloning site of the adenoviral shuttle plasmid pACCMVpLpASR- (kindly provided by Dr. Robert D. Gerard, University of Texas, Southwestern Medical Center) {Gomez-Fox, et al., J. Biol. Chem., 267:25129-25134 (1992)}. This plasmid contains the 5′ end of the adenovirus serotype 5 genome (map units 0 to 17) where the E1 region has been substituted with the human cytomegalovirus enhancer-promoter followed by the multiple cloning site from pAC19 and the polyadenylation region from SV40. The resulting plasmid was co-transfected with pJM17, a plasmid that contains the complete adenovirus 5 genome, into the human embryonic kidney cell line 293 using the calcium phosphate transfection method. Infectious viral particles containing the inserted hsp70 were generated by in vivo recombination in the 293 cells and were isolated as single plaques seven days later.

In addition, applicants also generated a control recombinant adenoviral construct that consisted of the pACCMVpLASR- plasmid without any insert. The isolated plaques were propagated in 293 cells for several passages to obtain high titer stocks. Viral particles were purified by CsCl ultracentrifugation. The titer of viral stocks was determined either by plaque assay or deproteination of an aliquot of the viral stock and amount of DNA determined by optical density {Barr, et al., Gene Therapy, 1:51-58 (1994)}.

### Protein Analysis

Cellular protein extracts were prepared from neonatal cardiomyocytes and H9c2 cells infected with adenoviral-hsp70i, the control adenoviral-SR- constructs or non-infected as previously described {Mestril, et al., J. Clin. Invest., 93:759-767 (1994)}. Protein concentration was determined by the Bradford Assay (BioRad Laboratories, Richmond, Calif.). Protein samples (40 μg each) were fractionated on an 8% SDS-polyacrylamide gel and electrotransferred onto nitrocellulose using a semi-dry electrotransfer apparatus (BioRad Laboratories). The nitrocellulose blots were reacted either with a monoclonal antibody C92F3A-5 (StressGen, Biotechnologies Corp., Victoria, BC) which binds specifically to the mammalian inducible HSP70 or with a polyclonal antiserum which binds to the COOH terminal of the mammalian HSP70s and HSP90s {Mehta, et al., Circ. Res., 63:512-517 (1988)}. Blots were subsequently reacted with biotinylated secondary antibodies and streptavidin-horseradish peroxidase-conjugated systems. (Vectastain, ABC kit; Vector Laboratories, Burlingame, Calif.) and developed with diaminobenzidine, tetrahydrochloride (DAB kit, Vector Laboratories).

### Indirect Immunofluorescence

Plates of infected and non-infected neonatal cardiomyocytes and H9c2 cells were washed twice with ice cold PBS and fixed with 100% ice cold methanol for 2 minutes. The fixed cells were then rehydrated with TBS containing 0.1% bovine serum albumin and reacted either with the monoclonal antibody against the inducible HSP70 (C92F3A-5) and subsequently developed with an ABC kit and VectorRed kit (Vector Laboratories) or a FITC-conjugated polyclonal antibody raised against the hexon coat protein of adenovirus (AB1056F, Chemicon International, Temecula, Calif.).

### Analytical Techniques

Creatine kinase (CK) activity was measured spectrophotometrically using a commercial CK kit (Sigma Immunochemicals, St. Louis, Mo.). CK activity release was expressed as the percent of the total CK activity present in each plate normalized by the amount of protein in each plate. Lactate dehydrogenase (LDH) activity was determined spectrophotometrically using a LDH test kit (Sigma). LDH activity released was expressed as the percent of the total LDH present in each plate normalized by the amount of protein present.

### Statistical Analysis

Results are expressed as the mean±standard error. Statistical significance was assessed by the Student's two-tailed test, unpaired t test and a probability value of <0.05 was considered significant.

### RESULTS

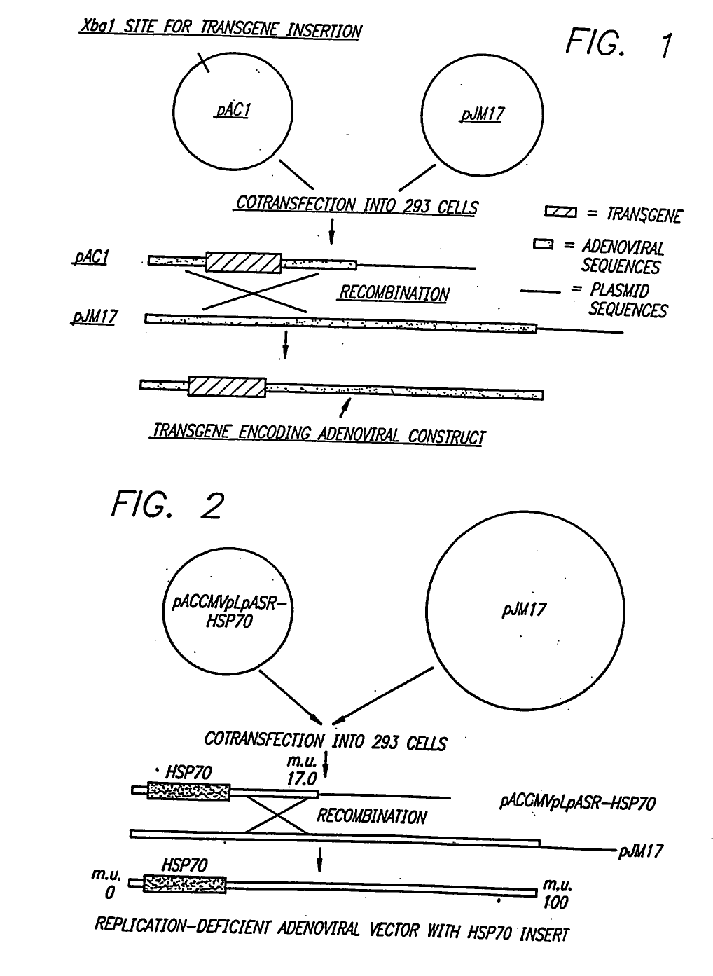

Several studies have shown that the sole expression of exogenous copies of hsp70 in cardiac tissue is sufficient to render the heart tolerant to ischemic injury {Marber, et al., J. Clin. Invest., 95:1446-1456 (1995); Plumier, et al., J. Clin. Invest., 95:1854-1860 (1995)}. This increased expression of the exogenous hsp70 does not occur only in cardiomyocytes, but also in non-myocytic cells, such as fibroblasts, endothelial and smooth muscle cell, present in the heart. Therefore, applicants were interested in introducing and expressing exogenous copies of hsp70i specifically in neonatal rat cardiomyocytes. For this purpose, applicants constructed a replication-deficient recombinant adenoviral vector containing the inducible rat hsp70 gene {Mestril, et al., Biochem. J., 298:561-569 (1994)}. The general strategy used to introduce a foreign gene into the E1 region of the replication-deficient adenoviral vector is represented schematically in FIG. 2 (see also Materials and Methods). In addition, the control adenoviral construct was generated using the same scheme with the exception that it lacks an insert.

In order to characterize the levels of infection and expression achieved with this adenoviral-hsp70i vector, protein extracts were prepared from neonatal rat cardiomyocytes 48 hours after infection. The protein extracts were examined by Western blot analysis. During the course of this study, three Western blots produced identical results. A representative Western blot was developed with a polyclonal antibody that binds to both HSP70 and HSP90. The Western blot has three lanes. The first lane contained proteins from non-infected myocytes. The second lane contained proteins from myocytes infected with the control adenoviral vector (adenoviral-SR) at a MOI of 10:1. The third lane contained proteins from myocytes infected with the adenoviral-hsp70i (MOI of 10:1). The Western blot showed that the adenoviral-hsp70i construct infected myocytes constitutively expressed a large amount of the exogenous hsp70i. To better examine the level of expression of the virally introduced hsp70i gene, applicants developed a second Western blot with a monoclonal antibody which binds specifically to the inducible HSP70. The second Western blot showed that while at a MOI of 1:1, the level of expression of HSP70 obtained with the adenoviral-hsp70i was lower than at a MOI of 10:1, it was still comparable to the normal expression of hsp70i in non-infected heat shocked cardiomyocytes (42° C., 60 minutes).

Since the control adenoviral vector (adenoviral-SR) lacked an insert, indirect immunofluorescence was used to detect infection by this adenoviral construct as well as that of the adenoviral-hsp70i construct in neonatal myocytes and H9c2 cells by using a polyclonal antibody that binds to the hexon assembly protein of adenovirus. The result was obtained of such an analysis on H9c2 cells that were infected with the adenoviral constructs 48 hours prior to fixation of the cells. Panels A and B of the indirect immunofluorescence were infected with the adenoviral-hsp70i construct (MOI of 1:1), panels C and D were infected with the control adenoviral-SR construct (MOI of 1:1) and panels E and F were non-infected cells. In panels A, C and E, cells were reacted with the monoclonal antibody against the inducible HSP70. In panels B, D and F, cells were reacted with the polyclonal antibody against the adenoviral hexon assembly protein. High levels of expression of hsp70i could only be observed in cells infected with the adenoviral-hsp70i and reacted with the monoclonal antibody specific to the HSP70i (panel A). While the polyclonal antibody against the adenovirus hexon assembly protein reacted with cells previously infected with either adenoviral-hsp70i or adenoviral-SR constructs (panels B and D), this indirect immunofluorescent analysis was done in three different occasions during the course of this study to monitor the reproducibility of the infection protocol. The results were identical in all three occasions. Identical results were obtained with neonatal rat myocytes.

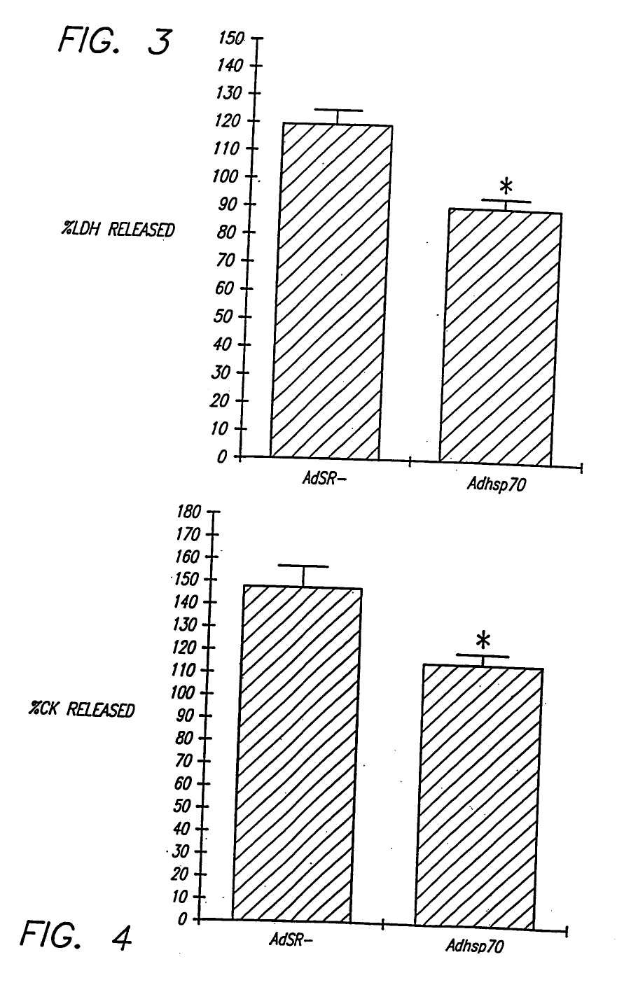

In order to test if the adenoviral transferred HSP70i preserves its protective function against stress, H9c2 cells were infected either with the adenoviral-hsp70i (designated “Adhsp70” in FIG. 3) (MOI of 1:1) or the adenoviral-SR (designated “AdSR-” in FIG. 3) (MOI of 1:1), and 48 hours later these cells were submitted to simulated ischemia. Applicants then measured the amount of lactate dehydrogenase activity released and remaining after simulated ischemia as a parameter of cellular damage. FIG. 3 shows the results obtained from six independent experiments. In FIG. 3, lactate dehydrogenase (LDH) released is expressed as a percentage of LDH released in control plates (infected but not submitted to simulated ischemia) which is taken as 100%. The amount of LDH released was calculated as the amount of LDH activity released, normalized by the amount of protein released (Units/mg) over the amount of total LDH activity normalized by the total amount of protein in each plate (total Units/mg). The p value is less that 0.05, indicating a statistically significant difference, and denoted by the “*” in FIG. 3. A similar series of experiments was performed with neonatal rat cardiomyocytes which were either infected with the adenoviral-hsp70i or the adenoviral-SR constructs (both at MOI of 1:1) and 48 hours later submitted to simulated ischemia. Creatine kinase activity released and remaining, after simulated ischemia, was measured to assess cellular damage to cardiomyocytes. FIG. 4 shows the results obtained in six independent experiments. In FIG. 4, the creatine kinase (CK) released is expressed as a percentage of CK released in control plates (infected but not submitted to simulated ischemia) which is taken as 100%. The amount of CK released was calculated as the amount of CK activity released, normalized by the amount of protein released (Units/mg) over the amount of total CK activity, normalized by the total amount of protein in each plate (total Units/mg). The p value is less than 0.05, indicating a statistically significant difference, and denoted by the “*” in FIG. 4. In both of the above sets of experiments, it was observed that the expression of the exogenous hsp70i seemed to render the cardiomyocyte and H9c2 cells more tolerant to cellular damage due to the simulated ischemia.

The above results show that not only are neonatal rat cardiomyocytes easily infectable by adenoviral vector particles, but also that this infection does not seem to have any deleterious effects on the myocytes. Both cardiomyocytes and the myogenic H9c2 cells were readily and reproducibly infectable by the above adenoviral constructs. One important point is that the infection of these cells with adenoviral vectors does not, in itself, elicited a stress response which can readily be noted by the lack of induction of the endogenous hsp70i gene upon infection with the control adenoviral-SR construct. In addition, both cardiomyocytes and H9c2 cells presented no deleterious effects two days after infection with adenoviral particles. Surprisingly, no apparent morphological changes or noxious effects to the cell were evident even in cells infected with the adenoviral-hsp70i construct that generated a large amount of HSP70i.

The sole presence of the exogenous hsp70i, in both neonatal cardiomyocytes and H9c2 cells, was capable of conferring protection against simulated ischemia in vitro to these cells (FIGS. 3 and 4). It should be noted that the level of protection obtained by the adenoviral-hsp70i construct in H9c2 cells was less than in the rat neonatal cardiomyocytes (FIGS. 3 and 4). One probable explanation for this difference in the level of protection may be due to the nature of these two cells. While the rat neonatal cardiomyocytes are non-dividing cells, the H9c2 cells are an established proliferating cell line. Therefore, at two days post-infection (the time needed to obtain sufficient expression of the exogenous protein, HSP70i), the number of adenoviral-hsp70i infected H9c2 cells may have been dilated out to a certain extent, resulting in a lower number of cells protected against simulated ischemia. Nonetheless, this would seem to prove that increased levels of HSP70i in the cardiomyocyte itself is able to enhance myocardial protection. Thus, the experiment supports the introduction of adenoviral constructs of the present invention into the hearts of animals to confer protection against myocardial ischemia.

We claim:

1. An isolated and purified recombinant adenoviral vector, said vector comprising:
an adenoviral genome from which the E1A/E1B genes have been deleted;
a transgene coding for a stress related factor which is a heat shock protein or the adenosine A3 receptor; and
a promoter operably linked to said transgene, wherein expression of the transgene is controlled by said promoter.

2. The vector of claim 1, wherein said stress related factor is selected from the group consisting of HSP70i, HSP27, HSP40, HSP60, and the adenosine A3 receptor.

3. The vector of claim 1, wherein said promoter is a CMV promoter.

4. The vector of claim 1, wherein said promoter is a ventricular myocyte-specific promoter.

5. A method of producing an isolated and purified recombinant vector of claim 1, comprising the steps of:
cloning a transgene coding for a stress related factor into a plasmid containing a promoter and a polylinker flanked by adenoviral sequences of the left end of the human adenovirus 5 genome from which the E1A/E1B genes have been deleted;
co-transfecting said plasmid into mammalian cells transformed with the E1A/E1B genes, with a plasmid which contains the entire human adenoviral 5 genome, and an additional insert making the plasmid too large to be encapsulated, whereby rescue recombination takes place between the transgene-inserted plasmid and the plasmid having the entire adenoviral genome so as to create a recombinant genome containing the transgene without the E1A/E1B genes, said recombinant genome being sufficiently small to be encapsulated;
identifying cells comprising recombinant vectors in cell cultures;
propagating the resulting recombinant vectors in mammalian cells transformed with the E1A/E1B genes; and
purifying the propagated recombinant vectors.

6. The method of claim 5, wherein said plasmid into which the transgene is cloned is plasmid pAC1 or plasmid ACCMVPLPA.

7. The method of claim 5, wherein said identification comprises the steps of:
monitoring transfected cells for evidence of cytopathic effect;
treating the cell supernatant from cell cultures showing a cytopathic effect with proteinase K, followed by phenol/chloroform extraction and ethanol precipitation to isolate viral DNA;
identifying cells producing recombinant vectors with PCR using primers complementary to the CMV promoter and primers complementary to adenoviral sequences; and
purifying recombinant vectors using two rounds of plaque purification.

8. The method of claim 5, wherein said purification comprises the steps of:
propagating the resulting recombinant vectors in cells transformed with the E1A/E1B genes to titers in the 10¹⁰-10¹² viral particles range;
purifying the propagated recombinant vectors by double CsCl gradient ultracentrifugation; and
filtering the purified recombinant vectors through sepharose columns.

9. The method of claim 5, wherein said stress related factor is selected from the group consisting of HSP70i, HSP27, HSP40, HSP60, and the adenosine A3 receptor.

10. A method of elevating the level of stress related factor in the myocardium of a patient, comprising delivering a replication-deficient viral vector to the myocardium of a patient, wherein said vector comprises a transgene encoding a stress related factor, and wherein delivery is by intracoronary injection into the lumen of one or both coronary arteries of said patient.

11. The method of claim 10, wherein the stress related factor is a heat shock protein or the adenosine A3 receptor.

12. The method of claim 10, wherein said stress related factor is selected from the group consisting of HSP70i, HSP27, HSP40, HSP60, and the adenosine A3 receptor.

13. The method of claim 10, wherein the stress related factor is a heat shock protein or the adenosine A3 receptor, and wherein the vector is an adenoviral vector comprising a gene encoding said heat shock protein or the adenosine A3 receptor.

14. The method of claim 10, wherein said patient has non-revascularized ischemic heart disease and wherein said vector is administered a plurality of days prior to non-cardiac surgery.

15. The method of claim 10, wherein said vector is delivered at the time of a diagnostic catheterization a plurality of days prior to complex percutaneous revascularization.

16. The method of claim 10, wherein said vector is delivered at the time of a diagnostic cardiac catheterization.

17. The method of claim 10, wherein said vector is delivered at the time of a diagnostic coronary angiography.

18. The method of claim 10, wherein said promoter is a CMV promoter.

19. The method of claim 10, wherein said promoter is a ventricular myocyte-specific promoter.

20. The method of claim 10, wherein said vector is delivered in the form of a viral stock having a final viral titer of 10¹⁰-10¹³ viral particles.
